(19) United States
(12) Patent Application Publication
(10) Pub. No.: US 2009/0176887 A1
(43) Pub. Date: Jul. 9, 2009

(54) Biocidal Compositions and Methods

(76) Inventors: Victor Vlasaty, Grayslake, IL (US); David Q. Cao, Chicago, IL (US)

Correspondence Address: MCANDREWS HELD & MALLOY, LTD, 500 WEST MADISON STREET, SUITE 3400, CHICAGO, IL, 60661, US

(21) Appl. No.: 12/364,290

(22) Filed: Feb. 2, 2009

Related U.S. Application Data
(63) Continuation of application No. PCT/US2007/075198, filed on Aug. 3, 2007.
(60) Provisional application No. 60/835,554, filed on Aug. 4, 2006.

(51) Int. Cl. A61K 31/14; A61P 31/00

(52) U.S. Cl. 514/642

(57) ABSTRACT

Biocidal compositions comprises an effective amount of a biocidal agent such as a quaternary ammonium compound or a blend of quaternary ammonium compounds and an effective amount of a cellular membrane disruptor. The synergy index of the biocidal agent and the cellular membrane disruptor is less than 1. The biocidal compositions can further comprise an effective amount of a chemical stabilizer and other optional components such as a nonionic surfactant. Methods for making and utilizing such biocidal compositions are also disclosed.

### RELATED APPLICATIONS

This application is a continuation of International application Serial No. PCT/US2007/075198 (International Publication No. WO 2008/019320), having an International filing date of Aug. 3, 2007. This PCT application claims priority to U.S. provisional patent application Ser. No. 60/835,554, filed Aug. 4, 2006. The entire specifications of the PCT and provisional applications referred to above are hereby incorporated by reference.

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

The present invention generally relates to one or more biocidal compositions and methods that contain or utilize one or more biocidal agents such as a quaternary ammonium compound (often referred to as a “quat”) or a blend of quaternary all onium compounds (often referred to as a blend of “quats”) and one or more cellular membrane disruptors. The biocidal compositions and methods may further contain or utilize one or more chemical stabilizers. The biocidal composition call be, for example, an antimicrobial or an antibacterial disinfectant composition.

Biocidal compositions, which may be, for example, germicides, antimicrobial or antibacterial blends, are widely used in different industries and in consumers' daily lives to inhibit or kill various microorganisms including, bacteria, viruses, or other susceptible pathogenic agents (collectively “biocidal targets”). Common classes of antimicrobials include, for example, chlorhexidine, alcohols, oxidizing agents (e.g., chlorine, iodine, iodophors, and peroxides), phenolics, quaternary ammonium compounds, and aldehydes.

A variety of quaternary ammonium compounds, including benzalkonium chloride and cetylpyridinium chloride, have been widely used since their introduction as germicides in 1935. The use of quats in disinfectant products remains popular primarily because of their relatively broad range of biocidal activity, low corrosivity, low toxicity, and low cost.

There are different types of physiological actions associated with quats. For example, it is known that quats attach to cell membranes and cause denaturing of cellular proteins therefore affecting the metabolic reactions of the cells. This action, in turn, adversely affects cell membrane permeability, causing vital substances to leak out of the cell which ultimately results in cell death.

Although quats are considered to be effective biocides, they do have some limitations. For example, when used alone their spectrum of activity may be considered limited in some applications. For instance, quats are generally not considered to be sporicidal and their activity against mycobacterium, some grain-negative bacteria, and certain non-enveloped viruses maybe minimal at normal use concentrations. Furthermore, the contact time required for quats to kill certain microorganisms can be relatively long (e.g., greater than 10 minutes). This slow rate of kill (“ROK”) may not be appropriate for some consumer, industrial or institutional applications.

Moreover, quats are sensitive to hard water (according to the U.S. Geological Survey Open-File Report 78-200, p. 436 (1977), 85% of U.S. homes have hard water), and often require a metal chelator, such as ethylenediaminetetracetate (EDTA) in the disinfectant formulation to obtain optimal biocidal efficacy. (See Buck, The Effects of Germicides on Microorganisms, www.infectioncolntroltoday.com/articles/191clean.hltml.) Further, it is known that metal chelators can be environmentally unfriendly or harmful. See http://www.laundry-alternative.com/detergentsinfo.html, F. Dietz, “Water Pollution by EDTA-a new challenge to water protection”, Belick Korrespondenz Abwasser, vol. 32, pp, 988-989 (1985).

It is also known that non-biocidal components (“inerts”) used in quat based biocidal products can impact biocidal performance. Efforts have therefore been made to formulate quats with inerts which actually enhance biocidal effectiveness by reducing the level of the quat(s) needed, widening their spectrum of activity, and/or improving their rates of kill. For example, some quat compositions mixed with low concentrations of alcohols have been advertised in the art as “tuberculocidal,” although such quats alone have no tuberculocidal activity. See Wayne et al., Finding the “Hidden Positive” in Tuberculosis Eradication Programs, Am Rev Respir Dis., 86:537-541 (1962); see also Smithwick et al., Use of cetylpyridinium chloride and sodium chloride for the decontamination of sputum specimens that are transported to the laboratory for the isolation of Mycobacterium tuberculosis, J. Clin. Microbiol., 1: 411-413 (1975).

Stepan Company (Northfield, Ill.) has also formulated quats with solvents (e.g., diethylene glycol monobutyl ether) to make proprietary disinfectant formulations that exhibit increased or improved biocidal activity. See U.S. Pat. No. 5,444,094 (Stepan Company). The manufacture or production of a dilutable solvent optimized concentrate product is however more difficult and costly due to the amount of solvent required. Additionally, the solvents used can make the resultant compositions incompatible with certain surfaces (e.g., polycarbonate surfaces) sought to be disinfected.

Likewise, some surfactants, especially certain nonionic surfactants, are believed to improve the efficacy of quat disinfectants. These materials have been formulated into disinfectant compositions as well. See Seymour S. Block, Disinfection, Sterilization, and Preservation, 5th ed., p. 287 (2001). However the concentration of surfactant must be controlled closely since high levels of surfactant can cause surface streaking and excessive foaming.

U.S. Pub. Pat. App. No. 2004/0058878 (Walker) discloses germicidal compositions with alleged enhanced activity towards killing microbiological spores and vegetative cells comprising certain quaternary ammonium compounds, phenolic compounds, monohydric alcohols, hydrogen peroxide, iodine, triclocarban, triclosan or combinations thereof with one or more spore coat opening agents. However, the reference describes the use and inclusion of metal chelation agents such as EDTA and (ethylenebis(oxyethylenenitrilo)) tetraacetic acid (EGTA), among others, as the spore coat opening agents. Such agents are not believed to be environmentally friendly and can be harmful under various conditions. Further, the Walker reference does not describe compositions or formulations that can improve the rate of kill associated with the germicidal agents used therein. Nor does it address stability issues related to some of the spore coat openers (e.g., the oxidation of ascorbic acid) which can and typically does reduce their effectiveness over time. Moreover, the Walker reference does not provide that its disclosed compositions can maintain the efficacy of the incorporated biocidal quats in hard water conditions without the use of metal chelation agents.

Thus, there is still a strong need for a quaternary based biocidal product that provides shorter contact times (i.e., faster rates of kill), a broader spectrum of activity, a better environmental profile, and/or a wider range of applications.

### BRIEF SUMMARY OF THE INVENTION

The presently described technology relates to biocidal compositions and methods that contain or utilize at least one biocidal agent such as a quat or a blend of quats and at least one cellular membrane disrupter. Preferably, the quat or blend of quats and the cellular membrane disruptor work or are capable of working together synergistically in a biocidal manner. The resulting biocidal compositions or methods exhibit an enhanced spectrum of activities and accelerated rates of kill. Further, the presently described technology can be utilized in a variety of environments (e.g., in the presence of hard water and/or proteinaceous soils), and can be more environmentally friendly (e.g., lower use concentrations as compared to other conventional biocidal compositions). Moreover, the biocidal composition of the present technology is preferably substantially free of metal chelation agents such as EDTA to improve environmental friendliness, while maintaining improved biocidal activity. Alternatively, the presently described technology can also provide biocidal products having a traditional biocidal efficacy profile while using lower amounts of the biocidal agents (e.g., a quat). Both dilutable concentrate and ready-to-use (RTU) biocidal products are envisaged in the scope, spirit and practice of the present technology.

In one aspect, the present technology provides a biocidal composition containing an effective amount of at least one biocidal agent and an effective amount of at least one cellular membrane disrupter. The cellular membrane disruptor can be a disulfide bond breaker such as ascorbic acid, glycolic acid, etc. The biocidal agent preferably comprises at least one quaternary ammonium compound. The biocidal composition preferably is substantially free of metal chelators and may further contain an effective amount of at least one chemical stabilizer such as sodium bisulfite. The biocidal composition can be provided, for example, via a solid, a powder, a gel, or a liquid form, and can be a dilatable concentrate or a ready-to-use product.

In another aspect of the present technology, a dilutable concentrate biocidal composition is provided, which comprises an effective amount of at least one quaternary ammonium compound (preferably a biocidal quaternary ammonium compound) and an effective amount of at least one disulfide bond breaker, wherein the synergy index of the quaternary ammonium compound and the disulfide bond breaker is less than 1.0. The dilatable concentrate biocidal composition can be formulated for making different ratios of dilutions (e.g., 1:256, 1:128, 1:64, 1:32, etc.).

For example, when the dilatable concentrate biocidal composition is utilized to make dilutions, it can contain from about 8.0% to about 14.0% of one or more quaternary ammonium compounds and from about 3.5% to about 6.5% of one or more disulfide bond breakers, (e.g., ascorbic acid), based on the total weight of the composition. The dilutable concentrate biocidal composition may further contain an effective amount of at least one chemical stabilizer (e.g., sodium bisulfite), and the composition is preferably substantially free of metal chelator agents (e.g., EDTA).

In a further aspect of the presently described technology, a ready-to-use biocidal composition is provided, which contains an effective amount of at least one quaternary ammonium compound and an effective amount of at least one disulfide bond breaker, wherein the synergy index of the quaternary ammonium compound and the disulfide bond breaker is less than 1.0. The ready-to-use biocidal composition can contain from about 0.01% to about 1.0% of one or more quaternary ammonium compounds, and from about 0.01% to about 0.5% of one or more disulfide bond breakers (e.g., ascorbic acid), based on the total weight of the biocidal composition. The ready-to-use biocidal composition can further contain an effective amount of at least one chemical stabilizer (e.g., sodium bisulfite), and the composition is preferably substantially free of metal chelator agents (e.g., EDTA). The pH of the ready-to-use biocidal composition is preferably adjusted to a range of from about 6.0 to about 9.0, alternatively from about 6.5 to 7.5, alternatively from about 6.5 to about 7.0.

In yet another aspect, the presently described technology provides one or more methods to make a biocidal composition in liquid form that comprises the steps of:

(1) adding a diluent into a container;

(2) adding at least one cellular membrane disruptor into the container; and then

(3) adding at least one biocidal quaternary ammonium compound into the container.

The method may further include the step of adding at least one chemical stabilizer into the container before the addition of the one or more cellular membrane disruptors. The cellular membrane disruptor can be ascorbic acid, and the chemical stabilizer can be sodium bisulfite, for example. Preferably, the ingredients added are mixed with minimal agitation between ingredient additions. The diluent can be, for example, water, a glycol, isopropanol, ethanol, methanol, or a combination thereof.

In yet a further aspect, the presently described technology provides a method of destroying, inhibiting or eliminating the growth of a biocidal target comprising the step of applying a biocidal composition of the present technology to a surface or a substrate for a contact time sufficient to destroy, inhibit, kill, reduce, or eliminate the biocidal target. The sufficient contact time can be less than about 10 minutes, alternatively about 5 minutes or less, alternatively about 3 minutes or less, or alternatively about 1 minute or less. Preferably, the contact time is less than about 10 minutes, more preferably from about 2 to about 5 minutes.

### BRIEF DESCRIPTION OF SEVERAL VIEWS OF THE DRAWINGS

[Not Applicable]

### DETAILED DESCRIPTION OF THE INVENTION

While the presently described technology will be described in connection with one or more preferred embodiments, it will be understood by those skilled in the art that the technology is not limited to only those particular embodiments. To the contrary, the presently described technology includes all alternatives, modifications, and equivalents as may be included within the spirit and scope of the appended claims.

It should be noted that, as used in this specification and the appended claims, the singular forms “a,” “an,” and “the” include plural referents unless the context clearly dictates otherwise.

As used herein, the term “biocidal” means capable of destroying, killing, neutralizing, reducing, eliminating, or inhibiting the growth of bacteria, microorganisms, germs, virus, spores, molds, yeasts, algae, and/or other susceptible pathogenic agents; biocidal can be, for example, antimicrobial, antibacterial, germicidal, sporicidal, antiviral, disinfectant, etc.

A “cellular membrane disruptor” in the present application means a substance or a combination of substances that can negatively impact cellular membrane integrity and render the cell(s) more permeable to a biocidal agent such as a positively charged biocidal quat.

As used herein, a “diluent” means a liquid or solid substance or mixture of substances that can be used as a delivery vehicle or carrier to prepare or dilute a biocidal composition of the present technology. A diluent can be, for example, water, a glycol, an alcohol, another polar solvent or any other liquid or solid that does not have a negative effect on the biocidal active materials.

A “disulfide bond breaker” in the present application means a substance or a combination of substances that is capable of reducing or breaking disulfide bonds.

A “ready-to-use” or “RTU” product, composition, or formulation in the present application refers to a product, composition, or formulation that is ready to be applied to articles or surfaces to be biocidally treated or disinfected.

A “dilatable,” “concentrate,” or “dilatable concentrate” product, composition, or formulation in the present application refers to a product, composition, or formulation that needs to be diluted with a diluent (e.g., water) in a ratio of, for example, 1:256, 1:128, 1:64, or 1:32, before it can be applied to articles, substrates, or surfaces to be biocidally treated or disinfected.

It has been surprisingly discovered that the biocidal effectiveness of quaternary ammonium compounds (“quats”) (e.g., allyl, dialkyl, or polymeric quats used singularly or in blends thereof) can be significantly enhanced by blending such compounds with one or more cellular membrane disruptors. One class of cellular membrane disruptors is disulfide bond breakers (e.g., ascorbic acid) that can breakdown or reduce the disulfide (e.g., S—S) bonds in cellular membranes such as those found in microorganisms.

It is also believed that the unique combination of cellular membrane disruptors and quats of the present technology can expand the traditional biocidal spectrum of activities of the quats and improve their rates of kill, thus reducing the required contact time to produce a biocidal effect. The compositions of the present technology are further believed to be capable of reducing or eliminating the growth of a wider range of organisms, including microorganisms, as compared to conventional quat-based formulations. Such a range can include, for example, green and blue-green algae, gram negative and gram positive bacteria, enveloped and non-enveloped viruses, molds, and yeasts. Collectively, these organisms could be called “biocidal targets.”

Additionally, it has been discovered that the combination of cellular membrane disruptors and quats of the present technology can eliminate the need to use environmentally questionable agents such as metal chelators (e.g., EDTA) to sustain the biocidal effectiveness of the quats in the presence of harsh environments (for example in the presence of hard water, soil, blood serum, and/or other organic contaminants).

Any quats can be used in the presently described technology. Examples of quats include, for example, allyl ammonium halides such as cetyl trimethyl ammonium bromide, alkyl aryl ammonium halides such as octadecyl dimethyl ammonium bromide, N-allyl pyridinium halides such as N-cetyl pyridinium bromide, and the like. One suitable type of quats includes, for example, those in which the molecules contain amine, ether or ester linkages such as octyl phenoxy ethoxy ethyl dimethyl benzyl ammonium chloride, N-(laurylcocoaminoformylmethyl)-pyridinium chloride, and the like. Another effective type of quats include, for example, those in which the hydrophobic radical is characterized by a substituted aromatic nucleus as the case of lauryloxyphenyltrimethyl ammonium chloride, cetylaminophenyltrimethyl ammonium methosulfate, dodecylphenyltrimethyl ammonium methosulfate, dodecylbelizyltrimethylammonium chloride, chlorinated dodecylbelizyltrimethyl ammonium chloride, and the like. Preferably, the quats utilized in the practice of the present technology exhibit biocidal activity or are biocidal in nature.

Although not wanting to be bound by any particular theory, it is believed that quats can adsorb to cell membranes of microorganisms (e.g., the biocidal targets listed above) and react chemically with negative charges carried by or therein to disrupt the cellular membrane and destroy the exposed microorganisms. It is further believed that such quats are not very effective against some microorganisms due to their inability to penetrate cellular membranes. Without intending to be bound by any particular theory, it is further believed that by combining quats with one or more cellular membrane disruptors, such a combination may increase the effectiveness of the quats, broaden their spectrum of activity, and accelerate their ROK. Further, some cellular membrane disruptors may be biocidal in nature, while some others may have no biocidal activity when used by themselves. It is also believed that the potential synergism between such components (i.e., the quat(s) and the cellular membrane disruptor(s)) can enhance the biocidal activity of each component, decrease the contact times needed to effectively kill or inhibit a biocidal target due to such synergism, and improve the rate of kill.

It has also been discovered that quats and cellular membrane disruptors in the composition of the present technology appear to form a synergistic combination having a synergy index of less than 1.0, alternatively not greater than about 0.6, alternatively not greater than about 0.51, as calculated by the industry accepted method described by S. C. Kull et al. in Mixtures of Quaternary Ammonium Compounds and Long-Chain Fatty Acids as Antifungal Agents, Applied and Environmental Microbiology, Vol. 9, pages 538-541 (1961). The Kull reference is incorporated herein by reference in its entirety, and more information about the method of calculating the synergy index will be provided below in the following examples. The synergistic activities of the components/compositions of the present technology illustrate the cooperative action of combining quats and cellular membrane disruptors of the present technology to yield a total biocidal effect which is greater than the sum of the biocidal effects of the quats and the cellular membrane disruptors when they are separately used.

One class of cellular membrane disruptors useful in the practice of the present technology is disulfide bond breakers. Examples of disulfide bond breakers include, but are not limited to, ascorbic acid (e.g., L- or D-ascorbic or mixtures thereof), lactic acid, gallic acid, ellagic acids, glycolic acid, thioglycolic acid, N-acetyl cysteine, and their respective esters, salts and derivatives thereof. Other examples of disulfide bond breakers can include 2-mercaptoethanol, aldehydes, quinone, polyphenol with up to hundreds of polymeric subunits including but not limited to phenol-rich polymers of flavonoids, and proaanthocyanidins including their free acid forms.

Other contemplated cellular membrane disruptors include, but are not limited to, solvents, oxidizers (e.g., peroxide), enzymes, and surfactants (e.g., the Zelec® series of products available from Stepan Company, Northfield, Ill.) that can negatively impact cellular membrane integrity. Examples of solvents include propylene glycol monomethyl ether (PGME), butyl carbitol, Steposol® DG solvent (available from Stepan Company), ethoxlated geraniol, and geraniol.

To achieve long-term storage stability and high temperature (e.g., at about 50° C.) stability, the biocidal compositions of the present technology may further contain at least one chemical stabilizer. The chemical stabilizer is especially preferred when the biocidal composition is a concentrated/dilutable product and/or when the cellular membrane disruptor utilized in the biocidal composition is vulnerable to hydrolysis or oxidation. The choice of the chemical stabilizer can depend on the type of the cellular membrane disruptor desired for a particular formulation of the present technology. Examples of suitable chemical stabilizers include, but are not limited to, sodium bisulfite, sodium borohydride, sodium metabisulfite, potassium metasulfite, sodium hydrosulfide, titanium sulfate, oxalic acid, and mixtures thereof.

For example, when ascorbic acid is used, a reducing agent such as sodium bisulfite (NaHSO₃) can be added to the biocidal composition as the chemical stabilizer. Again, although not wanting to be bound by any particular theory, the inclusion of the chemical stabilizer is believed to prevent discoloration (which can be caused by, for example, hydrolysis or oxidation of those cellular membrane disruptors that are sensitive), to prevent reduced biocidal effectiveness, and/or to enhance the storage and/or shelf-life, of the biocidal composition of the present technology. This in turn leads to reduced waste and enhanced cost savings. Potential hydrolysis of ascorbic acid (or another cellular membrane disruptor) can also be prevented or reduced by, for example, incorporating the biocidal composition into a glycol based or powdered formulation.

Preferably, the biocidal compositions of the present technology are substantially free of environmentally toxic metal chelation agents such as EDTA, EGTA, or nitrilotriacetic acid (NTA), although it is contemplated that the biocidal compositions can be made or used in hard water conditions. This outcome leads to a further advantage over conventional quat-based biocides and/or disinfectants.

The biocidal compositions of the present technology may include optional ingredients as known in the art. Such optional ingredients include, for example, surfactants (e.g., nonionic, cationic, or Zwitterionic surfactants), dyes, fragrances, preservatives, etc.

The biocidal compositions of the present technology can be prepared, for example, in a solid, gel, liquid or powdered form or any other suitable form using different delivery vehicles, and can be prepared as a ready-to-use or dilutable concentrate product. The delivery vehicles for liquid form compositions can be any diluent system known in the art. Examples of suitable diluents include, but are not limited to, water, glycols (preferably propylene glycol), alcohols (e.g., isopropanol, ethanol, methanol), other polar solvents known in the art, and mixtures thereof. Water is a preferred diluent of the presently described technology, and either de-ionized or regular tap water can be used. When glycols such as ethylene glycol are used, the diluent is preferably heated, for example, to from about 75° C. to about 150° C., alternatively from about 75° C. to about 100° C., when the biocidal actives are added to the diluent to improve solubility of the active materials.

The delivery vehicles or carriers for powdered form compositions of the present technology can also be called fillers. Any substance that is inert, dry, relatively low toxic, and cost effective can be used as the filler. Examples of suitable fillers include, but are not limited to, urea, dibasic calcium phosphate dehydrate, sodium sulfate, barium sulfate, calcite, calcium carbonate, wollastonite, calcium metasilicate, clay, aluminum silicate, magnesium aluminum silicate, hydrated alumina, silica, silicon dioxide, titanium dioxide, derivatives thereof, and mixtures thereof. The solid or gel form can be prepared using suitable delivery vehicles known in the art as well.

Standard blending equipment is acceptable for preparing the biocidal compositions of the present technology. Preparation, handling, and packaging precautions employed can be consistent with those established for quat-based formulations.

When making a liquid form biocidal composition of the present technology, preferably, the diluent (e.g., water or glycol) can be added into a blender or container followed by the addition of the chemical stabilizer, if one is used, then the cellular membrane disruptor, and finally the quat. Without intending to be bound by any particular theory, it is believed that by following this order of addition steps, oxidation of the cellular membrane disrupter can be reduced or avoided. Thorough mixing with minimal agitation is preferred between ingredient addition steps, which can further reduce oxidation of the cellular membrane disruptor. If a glycol is used in the diluent, the diluent is preferably heated to from about 75° C. to about 150° C., alternatively from about 75° C. to about 100° C. before the chemical stabilizer (e.g., sodium bisulfite) is added, and then the cellular membrane disruptor (e.g., ascorbic acid) is added at a temperature within this range. All components are preferably mixed until they are dissolved.

In accordance with at least one embodiment of the present technology, the biocidal composition can be an RTU product. The RTU product can contain from about 0.01% to about 1.0%, alternatively from about 0.02% to about 0.3%, alternatively from about 0.04% to about 0.12% of at least one quat or blend of quats, and from about 0.001% to about 5%, alternatively from about 0.01% to about 0.5%, alternatively from about 0.01% to about 0.1%, alternatively from about 0.02% to about 0.06% of at least one cellular membrane disruptor (e.g., ascorbic acid), based on the total weight of the biocidal composition. The RTU product can be in different forms (e.g., liquid, powder, solid, gel, etc.) using a variety of delivery vehicles available in the art. When the RTU product is in a liquid form, the diluent used to make the RTU product can be, for example, water, a glycol, or a mixture thereof.

Preferably, the RTU product can also contain from about 0.7% to about 1.5%, alternatively from about 0.85% to about 1.25%, alternatively from about 0.95% to about 1.1% of at least one chemical stabilizer (e.g., sodium bisulfite), based on the total weight of the biocidal composition. Optionally, the RTU product can contain from about 0% to about 0.15%, alternatively from about 0.01% to about 0.10%, alternatively from about 0.01% to about 0.05% of at least one surfactant, such as a nonionic surfactant, based on the total weight of the biocidal composition. Cationic and Zwitterionic surfactants can also be used in compositions of the present application. Other optional ingredients as known in the art including dyes, fragrances, preservatives, etc., can be added to the RTU product as well to help increase, for example, the stability and aesthetics of the products.

Preferably, the pH of the RTU product is adjusted to from about 6.0 to about 9.0, alternatively from about 6.5 to about 7.5, alternatively from about 6.5 to about 7.0. It has been discovered that an acidic RTU product can be ineffective against some biocidal targets such as gram-positive bacteria (e.g., Staphylococcus aureus). This problem can be reduced or eliminated by adjusting the pH of the biocidal composition of the present technology to the ranges as described above. Suitable amounts of alkaline agents such as sodium hydroxide, sodium carbonate, sodium bicarbonate, and the like can be used to adjust the pH of the RTU product to a desired value (e.g., pH 7).

In accordance with another embodiment of the present technology, the biocidal composition is a dilutable concentrate product. As defined above, a dilatable concentrate product is a product that needs to be diluted with a diluent (e.g., water) in a ratio of about, for example, 1:256, 1:128, 1:64, or 1:32 before it can be applied to articles or surfaces to be biocidally treated or disinfected. Depending on the intended dilution ratio, the concentration of actives in the dilutable concentrate product can vary. For a 1:128 dilutable concentrate product, for example, the dilutable concentrate composition can contain from about 8.0% to about 14.0%, alternatively from about 9.0% to about 12.5%, alternatively from about 10.0% to about 11.5% of at least one quat or blend of quats, and from about 1.0% to about 6.5%, alternatively from about 4.0 to about 6.0%, alternatively from about 4.5% to about 5.5% of at least one cellular membrane disruptor (e.g., ascorbic acid), based on the total weight of the biocidal composition. The dilutable concentrate product of the presently described technology is not limited to any particular form, and can use a variety of delivery vehicles available in the art. Water, either de-ionized or normal tap water, propylene glycol, or a mixture thereof, for example, can be used as the diluent.

Preferably, the 1:128 dilutable concentrate product, as an example of the present technology, can also contain from about 0.7% to about 1.5%, alternatively from about 0.85% to about 1.25%, alternatively from about 0.95% to about 1.10% of at least one chemical stabilizer (e.g., sodium bisulfite), based on the total weight of the biocidal composition. Optionally, but preferably, the 1:128 dilutable concentrate product can contain from about 1.0% to about 2.0%, alternatively from about 1.25% to about 1.8%, alternatively from about 1.4% to about 1.6% of at least one surfactant, such as a nonionic surfactant, based on the total weight of the biocidal composition.

Other optional ingredients as known in the art including dyes, fragrances, etc., can also be formulated into the dilutable concentrate products of the present technology. For example, the 1:128 dilutable concentrate product can contain from about 0.001% to 0.1% of a dye and from about 0.01% to about 0.5% of a fragrance.

If a dilutable product with 1:256, 1:64, 1:32, or another dilution ratio is intended, a person of ordinary skill in the art will be able to calculate such compositions of the present technology based on the above example of the proper ranges of the different components in a dilutable concentrate product for that particular embodiment.

In accordance with a further embodiment of the present technology, the biocidal compositions are provided for use in a powdered formulation. The powdered formulation can contain from about 8.0% to about 14.0%, alternatively from about 9.0% to about 12.5%, alternatively from about 10.0% to about 11.5% of at least one quat (e.g., a biocidal quat such as dialkyl, dimethylbenzyl, or ethylbenzyl type quat used singularly or in combination), and from about 1.0% to about 6.5%, alternatively from about 4.0% to about 6%, alternatively from about 4.5% to about 5.5% of at least one cellular membrane disruptor (e.g., ascorbic acid). Any substance that is inert and dry, exhibits relatively low toxicity, and is cost-effective can be used as a filler to make the powdered formulation. Examples of suitable fillers include, but are not limited to urea, sodium sulfate, dibasic calcium phosphate dehydrate, barium sulfate, calcite, calcium carbonate, wollastonite, calcium metasilicate, clay, aluminum silicate, magnesium aluminum silicate, hydrated alumina, silica, silicon dioxide, titanium dioxide, derivative thereof, and combinations thereof.

The powdered formulation can also contain from about 5.0% to about 6.0% of at least one nonionic surfactant (e.g., alcohol ethoxylate), from about 0.02% to about 4% of at least one alkaline agent (e.g., sodium carbonate), and/or from about 0.5% to about 1.5% of at least one chemical stabilizer (e.g., sodium bisulfite). The alkaline agent (such as sodium hydroxide, sodium carbonate, or sodium bicarbonate) is preferably included in the powdered formulation to assure that the pH of the powdered formulation when diluted for use is within the desired range (e.g., approximately 7.0).

A dehydrant/desiccant (e.g. precipitated silica) is preferably included in the powdered formulation to remove existing and/or potential available water from the formulation. Other optional ingredients as known in the art including, for example, dyes, fragrances, preservatives, etc., can be formulated into the powdered formulation of the present technology as well. For example, the powdered formulation can contain from about 0.001% to 0.1% of a dye and from about 0.01% to about 0.5% of a fragrance.

Besides a powdered disinfectant, a dilutable concentrate, or an RTU product as described above, the biocidal compositions of the present technology can also be made or incorporated into other forms of products such as a biocidal towelette (i.e., the use of the present technology on, in or incorporated with a substrate such as Rayon, polyester, polypropylene, or a mixture thereof), hand soap, water soluble sachets or packets, spray (e.g., aerosol spray or p-ump spray), foam, lotion, cream, wet wipe, etc. The biocidal compositions of the present technology are suitable for use as hard surface disinfectants for hospital, institutional, consumer, and veterinary applications; food and non-food contact sanitizers; preservatives; industrial water treatment biocides (e.g., recirculating cooling water system, air washers, simicides, algaecides), etc. The biocidal compositions can also be used for laundry sanitization/disinfection or odor control purposes, for example.

It is also contemplated that the quats of the present technology can be replaced by or used in combination with other biocidal agents such as aldehydes, phenolics, isothiazolines, alcohols, carbamates, halide compounds, peroxides, parabens, iodine, metals, peracids, carbonates, derivatives thereof, alternatives thereof, equivalents thereof or combinations thereof to produce further biocidal compositions of the presently described technology.

Although not wanting to be bound by any particular theory, with respect to spectrum of activity, it is believed that the biocidal compositions of the present technology can be capable of inhibiting, reducing or eliminating growth of a wide range of biocidal targets which may include, but are not limited to: green algae such as Chlorella vulgaris, Scenedesmus obliquus, Ulothrix lactuca, blue-green algae such as Oscillatoria lutea, Phormidium inundatum, Anabaena verrucosa, gram negative bacteria such as Campylobacter jejuni, Pseudomonas aeruginosa, Salmonella enterica, gram positive bacteria such as Mycobacterium tuberculosis, Staphylococcus aureus, Streptococcus pyogenes, Clostridium difficile, enveloped viruses such as Avian Influenza Virus, Hepatitis B Virus, West Nile Virus, Human Immunodeficiency Virus (HIV), non-enveloped viruses such as Adenovirus, Feline calicivirus, Hepatitis A Virus, Polio Virus, molds such as Penicillium marneffei, Aspergillus niger, Trichophyton mentographytes, and yeasts such as Candida albicans, Saccharomyces cerevisiae, Cryptococcus albidus. Although this listing of biocidal targets is not intended to be exhaustive, it will be appreciated by those skilled in the art that the biocidal compositions of the present technology exhibit an enhanced spectrum of activity over conventional quat-based biocidal formulations. Use of the biocidal compositions and methods of the present technology to inhibit, reduce or eliminate the growth of microbiological spores and vegetative cells is also contemplated. Further, as the following examples will demonstrate, the biocidal compositions of the present technology remain effective with reduced active concentrations, exhibit improved rates of kill, and offer shortened effective contact times. The effective contact time can be less than about 10 minutes, alternatively about 5 minutes or less, alternatively about 3 minutes or less, or alternatively about 1 minute or less. Preferably, the contact time is less than about 10 minutes, more preferably from about 2 to about 5 minutes.

The presently described technology and its advantages will be better understood by reference to the following examples. These examples are provided to describe specific embodiments of the present technology. By providing these specific examples, the inventors do not limit the scope and spirit of the present technology. It will be understood by those skilled in the art that the full scope of the presently described technology encompasses the subject matter defined by the claims appending this specification, and any alterations, modifications, alternatives, or equivalents of those claims.

### EXAMPLES

### Description of Materials Used in the Examples

In the examples, ABDAC refers to an allyl dimethyl benzyl chloride, which is a second generation quat as known in the art; DDAC refers to a diallyldimethyl ammonium chloride, which is a fourth generation quat as known in the art, ABDAC/DDAC refers to a blend of ABDAC and DDAC, which is a fifth generation quat mixture as known in the art.

The ABDAC/DDAC used in the examples is available from Stepan Company (Northfield, Ill.) as BTC® 885 (EPA Reg. No. 1839-113) (50% active).

The EDTA used in the examples is tetrasodium ethylenediaminetetraacetate (38% active), available from The Dow Chemical Company (Midland, Mich.) as Versene™ 100 or from Alzo Nobel Functional Chemicals, LLC (Huston, Tex.) as Dissolvine® E-39.

Biosoft® 25-7 (available from Stepan Company, Northfield, Ill.) is the nonionic surfactant used in the examples.

The bacteria used in the examples include:

Pseudomonas aeruginosa (P. aeruginosa): a highly resistant gram negative bacterium, which is often used to substantiate the efficacy of hospital type disinfectants, available from American Type Culture Collection (ATCC), Manassas, Va. as ATCC 15442;
Staphylococcus aureus (S. aureus): a gram positive bacterium, which is often used to substantiate the efficacy of limited or general disinfectants, available as ATCC 6538;
Escherichia coli (E. Coli): one of the main species of bacteria that live in the lower intestines of mammals, commonly used as a model organism for bacteria in general, available as ATCC 11229.

### Description of Methods Used in the Examples

Following is a general description of some methods used in the examples:

### Method to Determine Synergy

The synergistic activities of different components in a composition (control, conventional comparative, or of the present technology) is determined by the method described by Kull et al. in Allied Microbiology, Vol. 9, pages 538-541 (1961), which uses a synergy index to indicates whether synergy exists. A synergy index less than one (<1) indicates synergy; a synergy index of one (1) indicates additivity; a synergy index greater than one (>1) indicates antagonism.

When applied to Example 1 below, the synergy index of L-ascorbic acid and ADBAC/DDAC can be determined by the following equation:

Synergy Index=Qₐ/Q_{A}+Q_{b}/Q_{B},

wherein:
QA=the number of ppm needed for L-ascorbic acid alone to produce an endpoint,
QB=the number of ppm needed for ADBAC/DDAC alone to produce an endpoint,
Qa=the number of ppm of L-ascorbic acid needed when it is used in combination with ADBAC/DDAC to produce an endpoint,
Qb=the number of ppm of ADBAC/DDAC needed when the blend is used in combination with L-ascorbic acid to produce an endpoint,
further wherein ppm stands for part per million. The endpoint used in Example 1 is the Minimum Inhibitory Concentration (MIC), which represents the minim-alum concentration of a biocidal (e.g., antimicrobial) agent needed in a given culture medium to effectively inhibit microbial growth.

### Broth Dilution Method for Determining MIC

The Minimum Inhibitory Concentration (MIC) of a chemical against a certain bacterium is determined according to the broth dilution method described in National Committee for Clinical Laboratory Standards entitled, “Methods for Dilution Antimicrobial Susceptibility Test for Bacteria that Grow Aerobically” document M7-A2, 2^{nd} ed., 10:8, 1990. The broth dilution method determines the minimum concentration/maximum dilution of a biocidal agent that can inhibit the growth of a test organism. The organisms tested in Example 1 below include S. aureus, P. aeruginosa, and E coli.

### Use-Dilution Method for Determining Antimicrobial Efficacy

Biocidal efficacy of exemplary dilutable concentrate formulations (control, conventional comparative, or of the present technology) used in the examples are evaluated against S. aureus and/or P. aeruginosa. The testing was performed in accordance with the protocols outlined in Chapter 6 of “Official Methods of Analysis” of the Association of Official Analytical Chemists (AOAC) (17th Ed. 1998). More specifically, the protocols involved are AOAC Official Method 955.14 Testing Disinfectants Against Staphylococcus aureus (§ 6.2.04) and AOAC 964.02 Testing Disinfectants Against Pseudomonas aeruginosa (§ 6.02.06). The contents of Methods 955.14 and 964.02 and the methods referred therein (Methods 955.12, 955.14, and 955.14C) are all incorporated herein by reference in their entirety. The testing method is commonly referred to as the AOAC Use-Dilution Method.

The dilutable concentrates are tested in the presence of 400 ppm (as CaCO₃) synthetic hard water and 5% organic soil load.

The efficacy of a biocidal composition according to the Use-Dilution Method can be indicated by the ratio of the number of tested carriers that show growth of the organisms on them over the total number of tested carriers bearing the test organisms that are treated with the test biocidal composition for a pre-selected contact time. For example, a result of “0/60” indicates that the test organisms show growth on zero (0) of the 60 carriers bearing the test organisms that are treated with the tested biocidal composition for the pre-selected contact time (e.g., 10 or 5 minutes). The “0/60” result shows that the growth of the microorganisms has been 100% inhibited. On the other hand, a “3/60” result shows that the organisms grow on three (3) of the 60 tested carriers and the growth inhibition rate is only 95%. In the examples, the standard for efficacy of biocidal compositions used are as follows:

Effective disinfectant: requires greater than 98% growth inhibition;

Marginally effective disinfectant: requires 95-98% growth inhibition;

Ineffective disinfectant: less than 95% growth inhibition.

### Example 1

### Determination of Synergistic Activity Between Ascorbic Acid and ADBAC/DDAC

In this example, standard MIC values of ascorbic acid, ADBAC/DDAC, and a 1:1 blend of ascorbic acid and ADBAC/DDAC were determined against E coli, S. aureus, and P. aeruginosa according to the broth dilution method introduced above. The pH1 of each composition containing the tested compound(s) was adjusted to about 7. The synergy index of the 1:1 blend of ascorbic acid and ADBAC/DDAC against each of the biocidal targets was then calculated according to the method as described above. The results are shown in Table 1 below.

TABLE 1

| | | MIC | |
|---|---|---|---|
| Compound | Biocidal Target | (ppm, active) | Synergy Index |
| L-Ascorbic Acid | E coli, | >10,000 | N/A |
| | S. aureus | >10,000 | |
| | P. aeruginosa | >10,000 | |
| ADBAC/DDAC | E coli, | 10 | N/A |
| | S. aureus | 1 | |
| | P. aeruginosa | 100 | |
| 1:1 Blend of L- | E coli, | 5:5 | ≦0.50 |
| Ascorbic Acid and | S. aureus | 0.5:0.5 | ≦0.50 |
| ADBAC/DDAC | P. aeruginosa | 50:50 | ≦0.51 |

The above synergy index values calculated for each test organism clearly demonstrate synergistic qualities existing between ascorbic acid and ADBAC/DDAC, which are biocidal quaternary ammonium compounds of the presently described technology.

### Example 2

### Comparative Study of Disinfectant Efficacy of Dilutable Concentrate Formulations

This example demonstrates how the disinfectant efficacy of a conventional ADBAC/DDAC based formulation can be enhanced by incorporating the presently described technology into the formula and eliminating the use of EDTA. The two organisms (biocidal targets) used in this example were (1) P. aeruginosa, a highly resistant gram negative bacterium, which is often used to substantiate the efficacy of hospital type disinfectants, and (2) S. aureus, a gram positive bacterium, which is often used to substantiate the efficacy of general disinfectants/biocides.

The formulations of the compositions tested included:

Formula A: a comparative essentially neutral (pH=approximately 7.0) disinfectant cleaner commercially available from Stepan Company (Northfield, Ill.) as BTC® 885 Neutral Disinfectant Cleaner-128, which contained 10.9% of ADBAC/DDAC, 1.5% of a nonionic surfactant, and 5.0% of EDTA, all based on the total weight of the composition.
Formula B: a comparative essentially neutral (pH=approximately 7.0) composition containing 1.5% of a nonionic surfactant, and 5.0% of EDTA, both based on the total weight of the composition. Formula B contained no quats.
Formula C: a comparative essentially neutral (pH=approximately 7.0) composition containing 5% of ascorbic acid, 1.5% of a nonionic surfactant, and 5% of EDTA, all based on the total weight of the composition. Formula C contained no quats.
Formula D: a comparative essentially neutral (pH=approximately 7.0) composition containing 5% of ascorbic acid and 1.5% of a nonionic surfactant, both based on the total weight of the composition. Formula D did not contain any quat and EDTA.
Formula E: a pH 4.5 composition of the present technology containing 10.9% of ADBAC/DDAC, 5% of ascorbic acid, and 1.5% of a nonionic surfactant, all based on the total weight of the composition. Formula E contained no EDTA.
The diluent for all Formulas A-E was deionized (DI) water, which made up the balance of each composition.

More detailed information about the formulations of Formulas A-E are shown in Table 2 below.

TABLE 2

| | Formula A | Formula B | Formula C | Formula D | Formula E |
|---|---|---|---|---|---|
| Ingredients | (wt %) | (wt %) | (wt %) | (wt %) | (wt %) |
| DI Water | 70.64 | 91.40 | 91.40 | 91.40 | 71.80 |
| Versene ™ | 5.00 | 5.00 | 5.00 | 0.00 | 0.00 |
| 100 | | | | | |
| Biosoft ® | 1.50 | 1.50 | 1.50 | 1.50 | 1.50 |
| 25-7 | | | | | |
| Citric Acid | 0.65 | 0.00 | 0.00 | 0.00 | 0.00 |
| BTC ® 885 | 21.70 | 0.00 | 0.00 | 0.00 | 21.70 |
| (50% | | | | | |
| Active) | | | | | |
| Dye | 0.01 | 0.00 | 0.00 | 0.00 | 0.00 |
| Fragrance | 0.50 | 0.00 | 0.00 | 0.00 | 0.00 |
| Sodium | 0.00 | 2.10 | 2.10 | 2.10 | 0.00 |
| Carbonate | | | | | |
| Ascorbic | 0.00 | 0.00 | 5.00 | 5.00 | 5.00 |
| Acid | | | | | |
| Total | 100.00 | 100.00 | 105.00 | 100.00 | 100.00 |

The disinfectant efficacy of Formulas A-E were tested according to the AOAC Use-Dilution Method introduced above. Formulas A-E were all 1:128 dilutable concentrate compositions, that were diluted in a synthetic hard water (400 ppm as CaCO₃) in the presence of 5% by weight organic soil load at a 1 to 128 ratio to make dilutions for testing (sometimes called “at use dilutions”). The pH of each dilution was approximately 6.5. The contact time selected for these particular tests was five (5) minutes. The test results are shown in Table 2 below.

TABLE 3

| Formulas | | |
|---|---|---|
| (1:128 Dilution) | Efficacy Results | Description of Efficacy |
| Formula A | 5/40, P. aeruginosa | Ineffective disinfectant |
| Formula B | 40/40, P. aeruginosa | Ineffective disinfectant |
| Formula C | 40/40, P. aeruginosa | Ineffective disinfectant |
| Formula D | 40/40, P. aeruginosa | Ineffective disinfectant |
| Formula E | 0/60, P. aeruginosa | Effective disinfectant |
| | 0/20, S. aureus | |

The comparative study illustrates that the composition comprising a blend of quats and ascorbic acid (i.e., at least one exemplary embodiment of the present technology) demonstrated superior biocidal properties as compared to formulations not containing the described blend.

The tests further demonstrate that the biocidal performance of a typical commercially available quat-based disinfectant formulation, which was represented by Formula A in this example, can be enhanced by incorporating the presently described technology. Formula A normally requires at least 10 minutes of contact time to be an effective disinfectant biocide. Therefore, Formula E acts faster, i.e., has a faster rate of kill (ROK) and achieves a shortened required contact time. Moreover, Formula E demonstrated a broader spectrum of biocidal activity in the described tests than Formula A, and eliminated the need for environmentally questionable compounds such as EDTA, which is required by Formula A.

### Example 3

### Comparative Study of the Shelf-Life/Stability of Dilutable Concentrate Formulations

The purpose of this experiment was to study the shelf-life/stability of two 1:128 dilutable concentrate compositions over a prolonged period of time (approximately 2 months). The two compositions studied were:

Formula F: a two month old essentially neutral (pH=approximately 7.0) composition of Formula E tested above that contained 10.9% of ADBAC/DDAC, 5% of ascorbic acid, and 1.5% of a nonionic surfactant, all based on the total weight of the composition.
Formula G: a two month old essentially neutral (pH=approximately 7.0) composition containing 10.9% of ADBAC/DDAC, 5% of ascorbic acid, 1.5% of a nonionic surfactant, and 1% sodium bisulfite, all based on the total weight of the composition.

The disinfectant efficacy of Formulas F and G were tested according to the AOAC Use-Dilution Method introduced herein. The same as in Example 2, Formulas F and G were diluted in a synthetic hard water (400 ppm as CaCO₃) in the presence of 5% by weight organic soil load at a 1 to 128 ratio to make dilutions for testing. The pH of the “at use dilutions” was about 6.5. The contact time selected for these tests was five (5) minutes. The test results are shown in Table 3 below.

TABLE 4

| Formulas | | |
|---|---|---|
| (1:128 Dilution) | Efficacy Results | Description of Efficacy |
| Formula F | 4/60, P. aeruginosa | Ineffective disinfectant |
| | 3/20, S. aureus | |
| Formula G | 0/30, P. aeruginosa | Effective disinfectant |
| | 0/30, S. aureus | |

The results illustrate that the disinfectant efficacy of Formula F with 5% ascorbic acid, but containing no sodium bisulfite, which was a two month old aged Formula E, decreased significantly in approximately two months as compared to the freshly prepared Formula E tested in Example 2. The loss of disinfectant efficacy is believed to be attributed to the hydrolysis of ascorbic acid to the pigmented compound, dehydroascorbic acid. The results also show that the addition of 1% sodium bisulfite in Formula G inhibits the pigmentation of ascorbic acid and preserves Formula G's disinfectant efficacy over an extended period of time.

This example demonstrates the importance of including a chemical stabilizer when the cellular membrane disruptor used in the biocidal composition of the present technology is not chemically stable, in order to extend the shelf-life/stability and/or to improve the high temperature stability of the biocidal composition.

### Example 4

### Comparative Study of Ready-to-Use Compositions

In this example, three Ready-to-Use (RTU) compositions were studied for their biocidal activities against the gram-positive bacterium S. aureus and the gram-negative bacterium P. aeruginosa. The three compositions were:

Formula H: a comparative RTU composition containing approximately 400 ppm ADBAC/DDAC with an adjusted pH of about 7.0;
Formula I: an RTU composition containing approximately 400 ppm ADBAC/DDAC and 380 ppm ascorbic with an unadjusted pH of around 4.5;
Formula J: an RTU composition containing approximately 400 ppm ADBAC/DDAC and 380 ppm ascorbic with the pH adjusted to about 7.0.
Deionized water was used as the diluent, which made up the balance of each composition.

The results are shown in Table 5 below:

TABLE 5

| | Efficacy Results (contact | |
|---|---|---|
| Formula (RTU) | time) | Description of Efficacy |
| Formula H | 0/20, P. aeruginosa (10 min) | Effective disinfectant |
| | 1/20, S. aureus (10 min) | Marginally effective |
| | | disinfectant |
| Formula I | 0/20, P. aeruginosa (5 min) | Effective disinfectant |
| | 20/20, S. aureus (10 min) | Ineffective disinfectant |
| Formula J | 0/20, P. aeruginosa (5 min) | Effective disinfectant |
| | 0/20, S. aureus (10 min) | Effective disinfectant |

First, comparing Formula J of the present technology to Formula H of the prior art, Formula J acted faster against P. aeruginosa and was more effective against S. aureus than Formula H. This demonstrates that the biocidal composition of the present technology has superior biocidal efficacy, broader spectrum of activity, and enhanced rate of kill, and achieves a shorter effective contact time as compared to Formula H.

On the other hand, comparing Formula I to Formula J, the results illustrate that Formula I is not effective against S. aureus. The difference between Formulas I and J is that the pH of the former is not adjusted, while the latter is adjusted to a neutral pH (about 7.0). This outcome demonstrates that at least for some RTU compositions of the present technology, it is important to adjust pH of the compositions to improve their efficacy.

The present technology is now described in such full, clear, and concise terms as to enable a person skilled in the art to which it pertains, to practice the same. It is to be understood that the foregoing describes preferred embodiments of the present technology and that modifications may be made therein without departing from the spirit or scope of the present technology as set forth in the claims.

What is claimed is:

1. A biocidal composition comprising an effective amount of at least one biocidal agent and an effective amount of at least one cellular membrane disruptor.

2. The biocidal composition of claim 1, wherein the at least one cellular membrane disruptor is at least one disulfide bond breaker.

3. The biocidal composition of claim 2, wherein the disulfide bond breaker is selected from the group consisting of ascorbic acid, lactic acid, gallic acid, ellagic acids, glycolic acid, thioglycolic acid, N-acetyl cysteine, derivatives thereof, and mixtures thereof.

4. The biocidal composition of claim 2, wherein the disulfide bond breaker is selected from the group consisting of 2-mercaptoethanol, quinone, polyphenols, proanthocyanidins, aldehydes, derivatives thereof, and mixtures thereof.

5. The biocidal composition of claim 1, wherein the biocidal agent comprises at least one quaternary ammonium compound.

6. The biocidal composition of claim 5, wherein the composition is substantially free of metal chelators.

7. The biocidal composition of claim 5, further comprising an effective amount of at least one chemical stabilizer.

8. The biocidal composition of claim 7, wherein the chemical stabilizer is selected from the group consisting of sodium bisulfite, sodium metabisulfite, potassium metasulfite, sodium hydrosulfide, titanium sulfate, oxalic acid, derivatives thereof, and mixtures thereof.

9. The biocidal composition of claim 5, wherein the at least one cellular membrane disruptor is at least one disulfide bond breaker.

10. The biocidal composition of claim 9, wherein the at least one disulfide bond breaker comprises ascorbic acid.

11. The biocidal composition of claim 10, further comprising an effective amount of sodium bisulfite.

12. The biocidal composition of claim 1, wherein the synergy index of the at least one biocidal agent and the at least one cellular membrane disruptor is less than about 1.0.

13. The biocidal composition of claim 12, wherein the synergy index is not greater than about 0.51.

14. The biocidal composition of claim 5, wherein the composition is in a powder form, comprising:
from about 8.0% to about 14.0% of the at least one quaternary ammonium compound; and
from about 1.0% to about 6.0% of the at least one cellular membrane disruptor, based on the total weight of the biocidal composition.

15. The biocidal composition of claim 14, further comprising from about 1.0% to about 6.0% of at least one surfactant.

16. The biocidal composition of claim 15, wherein the surfactant is a nonionic surfactant.

17. The biocidal composition of claim 14, further comprising from about 0.5% to about 1.5% of at least one chemical stabilizer.

18. The biocidal composition of claim 17, wherein the at least one chemical stabilizer is selected from the group consisting of sodium bisulfite, sodium metabisulfite, potassium metasulfite, sodium hydrosulfide, titanium sulfate, oxalic acid, derivatives thereof, and mixtures thereof.

19. The biocidal composition of claim 14, further comprising from about 0.01% to about 4.0% of at least one alkaline agent.

20. The biocidal composition of claim 19, wherein the alkaline agent is sodium hydroxide, sodium carbonate, sodium triphosphate, sodium methasilicate, sodium silicate, or a mixture thereof.

21. A dilutable concentrate biocidal composition comprising an effective amount of at least one quaternary ammonium compound and an effective amount of at least one disulfide bond breaker, wherein the synergy index of the at least one biocidal quaternary ammonium compound and the disulfide bond breaker is less than 10.

22. The dilutable concentrate biocidal composition of claim 21, wherein the dilutable concentrate biocidal composition is used to male a 1:256, 1:128, 1:64, or 1:32 dilution.

23. The dilutable concentrate biocidal composition of claim 22, wherein the dilutable concentrate biocidal composition is 1:128 dilutable, wherein the dilutable concentrate biocidal composition comprises from about 8.0% to about 14.0% of the at least one biocidal quaternary ammonium compound; and from about 1.0% to about 6.5% of the at least one disulfide bond breaker, based on the total weight of the dilutable concentrate biocidal composition.

24. The dilutable concentrate biocidal composition of claim 21, wherein the at least one disulfide bond breaker is selected from the group consisting of ascorbic acid, lactic acid, gallic acid, ellagic acids, glycolic acid, thioglycolic acid, N-acetyl cysteine, derivatives thereof, and mixtures thereof.

25. The dilutable concentrate biocidal composition of claim 21, further comprising an effective amount of at least one chemical stabilizer.

26. The dilatable concentrate biocidal composition of claim 25, wherein the at least one chemical stabilizer is selected from the group consisting of sodium bisulfite, sodium metabisulfite, potassium metasulfite, sodium hydrosulfide, titanium sulfate, oxalic acid, derivative thereof, and mixtures thereof.

27. The dilutable concentrate biocidal composition of claim 25, wherein the dilutable concentrate biocidal composition is 1:128 dilatable, wherein the at least one chemical stabilizer is present in the amount of from about 0.5% to about 1.5%, based on the total weight of the dilutable concentrate biocidal composition.

28. The dilutable concentrate biocidal composition of claim 21, further comprising an effective amount of at least one nonionic surfactant.

29. The dilutable concentrate biocidal composition of claim 21, wherein the composition is substantially free of metal chelators.

30. A ready-to-use biocidal composition comprising an effective amount of at least one quaternary ammonium compound and an effective amount of at least one disulfide bond breaker, wherein the synergy index of the at least one quaternary ammonium compound and the at least one disulfide bond breaker is less than about 1.0.

31. The ready-to-use biocidal composition of claim 30, comprising from about 0.01% to about 1.0% of the at least one biocidal quaternary ammonium compound; and from about 0.01% to about 5% of the at least one disulfide bond breaker, based on the total weight of the ready-to-use biocidal composition.

32. The ready-to-use biocidal composition of claim 30, wherein the at least one disulfide bond breaker comprises ascorbic acid.

33. The ready-to-use biocidal composition of claim 32, further comprising from about 0.05% to about 1.5% of sodium bisulfite.

34. The ready-to-use biocidal composition of claim 30, further comprising an effective amount of at least one chemical stabilizer.

35. The ready-to-use biocidal composition of claim 30, wherein the ready-to-use biocidal composition has a pH in the range of from about 6.0 to about 9.0.

36. The ready-to-use biocidal composition of claim 30, wherein the ready-to-use biocidal composition has a pH in the range of from about 6.5 to about 7.5.

37. The ready-to-use biocidal composition of claim 30, wherein the ready-to-use biocidal composition has a pH in the range of from about 6.5 to about 7.0.

38. The ready-to-use biocidal composition of claim 30, wherein the ready-to-use biocidal composition is substantially free of metal chelators.

39. A method for destroying or inhibiting growth of a biocidal target comprising the step of applying to a surface or substrate a biocidal composition comprising an effective amount of at least one biocidal agent and an effective amount of at least one cellular membrane disruptor.

40. The method of claim 39, wherein the composition is applied to the surface or the substrate in the form of a spray, liquid, powder, foam, lotion, cream, gel, solid, hand soap, water soluble packet administered in a diluent, sachet, towelette, or wet wipe.

41. The method of claim 39, further comprising the step of maintaining contact of the biocidal composition to the surface or the substrate for an effective period of time to destroy or inhibit the growth of the biocidal target.

42. The method of claim 41, wherein the effective period of time is less than about 10 minutes.

43. The method of claim 41, wherein the effective period of time is about 5 minutes or less.

44. The method of claim 41, wherein the effective period of time is about 3 minutes or less.

45. The method of claim 41, wherein the effective period of time is about 1 minute or less.
